(19) United States
(12) Patent Application Publication
(10) Pub. No.: US 2021/0247378 A1
(43) Pub. Date: Aug. 12, 2021

(54) NANOPORE DEVICE AND METHODS OF DETECTING AND CLASSIFYING CHARGED PARTICLES USING SAME

(71) Applicant: PALOGEN, INC., Palo Alto, CA (US)

(72) Inventors: Bita Karimirad, Seoul (KR); Kyung Joon Han, Palo Alto, CA (US)

(21) Appl. No.: 17/172,553

(22) Filed: Feb. 10, 2021

Related U.S. Application Data
(60) Provisional application No. 62/972,415, filed on Feb. 10, 2020.

(51) Int. Cl. G01N 33/487; C12Q 1/6827

(52) U.S. Cl. CPC G01N 33/48721; C12Q 1/6827

(57) ABSTRACT

A method of determining an oligonucleotide methylation percentage includes providing a 3D nanopore device having top and bottom chambers, and a 3D nanochannel array disposed therein. The method also includes purifying an oligonucleotide, and functionalizing the 3D nanochannel array by coupling an oligonucleotide probe. The method further includes forming an oligonucleotide solution having a known concentration, and adding the oligonucleotide solution to the top and bottom chambers. Moreover, the method includes placing top and bottom electrodes in the top and bottom chambers respectively, applying an electrophoretic bias between the top and bottom electrodes, applying a selection bias across first and second gating nanoelectrodes, applying a sensing bias through a sensing nanoelectrode in the 3D nanopore device. In addition, the method includes detecting an output current from the sensing nanoelectrode, and analyzing the output current from the sensing nanoelectrode to determine a methylation percentage of the oligonucleotide.

### CROSS-REFERENCE TO RELATED APPLICATION

This application claims priority to U.S. Provisional Application No. 62/972,415, filed on Feb. 10, 2020 under attorney docket number PAL.30009.00 and, entitled “NANOPORE DEVICE AND METHODS OF DETECTING AND CLASSIFYING CHARGED PARTICLES USING SAME,” the contents of which are hereby expressly and fully incorporated by reference in their entirety, as though set forth in full. This application includes subject matter similar to the subject matter described in co-owned U.S. Provisional Patent Application Ser. No. 62/566,313, filed on Sep. 29, 2017 under attorney docket number 165-101USIP and entitled “MANUFACTURE OF THREE DIMENSIONAL NANOPORE DEVICE”; U.S. Provisional Patent Application Ser. No. 62/593,840, filed on Dec. 1, 2017 under attorney docket number BTL.30002.00 and entitled “NANOPORE DEVICE AND METHOD OF MANUFACTURING SAME”; U.S. Provisional Patent Application Serial Number U.S. Provisional Patent Application Ser. No. 62/612,534, filed on Dec. 31, 2017 under attorney docket number BTL.30003.00 and entitled “NANOPORE DEVICE AND METHODS OF ELECTRICAL ARRAY ADDRESSING AND SENSING”; U.S. Provisional Patent Application Ser. No. 62/628,214, filed on Feb. 8, 2018 under attorney docket number BTL.30004.00 and entitled “BIOMEMORY FOR NANOPORE DEVICE AND METHODS OF MANUFACTURING SAME”; U.S. Utility patent application Ser. No. 16/147,362, filed on Sep. 26, 2018 under attorney docket number BTL.20001.00 and entitled “NANOPORE DEVICE AND METHOD OF MANUFACTURING SAME”; U.S. Utility patent application Ser. No. 16/237,570, filed on Dec. 31, 2018 under attorney docket number BTL.20003.00 and entitled “NANOPORE DEVICE AND METHODS OF ELECTRICAL ARRAY ADDRESSING AND SENSING”; U.S. Provisional Patent Application Ser. No. 62/802,459, filed on Feb. 7, 2019 under attorney docket number BTL.30004.01 and entitled “BIOMEMORY FOR NANOPORE DEVICE AND METHODS OF MANUFACTURING SAME”; U.S. Provisional Patent Application Ser. No. 62/826,897, filed on Mar. 29, 2019 under attorney docket number BTL.30006.00 and entitled “NANOPORE DEVICE AND METHODS OF BIOSYNTHESIS USING SAME”; U.S. Utility patent application Ser. No. 16/524,033, filed on Jul. 27, 2019 under attorney docket number PAL.20005.00 and entitled “NANOPORE DEVICE AND METHODS OF DETECTING CHARGED PARTICLES USING SAME”; and U.S. Provisional Patent Application Ser. No. 62/874,766, filed on Jul. 16, 2019 under attorney docket number PAL.30007.00 and entitled “NANOPORE DEVICE AND METHODS OF DETECTING AND CLASSIFYING CHARGED PARTICLES USING SAME.” The contents of the above-mentioned applications are fully incorporated herein by reference as though set forth in full.

### FIELD OF THE INVENTION

The present invention relates generally to systems and devices for characterizing epigenetic alterations, and methods of detecting methylation patterns in genomes using such systems and devices. In particular, the present invention relates to nanopore sensors for detecting methylation patterns. The disclosed nanopore sensors facilitate characterization of epigenetic alterations by characterizing methylation patterns in genome-derived oligonucleotides (e.g., detecting DNA methylation in genome-derived oligonucleotides).

### BACKGROUND

Early cancer detection and treatment can save millions of lives. Accordingly, there is a need for a device (e.g., a bedside/point of care detection system) and method for affordable, rapid, accurate, and early detection of epigenetic alterations in specific genes in a genome.

The etiology of cancer includes many types of genetic changes that can lead to various alterations in cell functions. In addition to genetic mutations, the etiology of cancer includes epigenetic changes, which is directly related to the gene expression and cancer. Detecting epigenetic changes can provide effective screening techniques for cancer detection, and subsequent treatment and cure by therapeutic intervention that conforms to the particular early detected cancer.

Cytosine poly guanine island (“CpG island”) methylation, histone modifications, and reorganization of chromatin various epigenetic mechanisms that regulate the activation and silencing of genes. DNA methylation is an epigenetic mechanism that can control DNA transcription and replication. Methylation patterns during tissue specific cell type differentiation from stem cells, are conserved during subsequent cell division to maintain the specific cell type in newly formed tissue.

Many genes can be activated or silenced resulting in carcinogenesis. While some mutations result in gene silencing, a significant extent of carcinogenic gene silencing is the result of alterations in DNA methylation. DNA methylation alteration in multiple CpG sites in a CpG island, especially in protein promotor regions, can lead to cancer via silencing of cancer reducing genes (e.g., error correction enzymes).

Even the silencing of the genes caused by other processes can be stabilized when the gene silencing is followed by promotor methylation in the CpG islands. Methylation is very effective in gene silencing. For instance, hypermethylation of a CpG island in a promotor region is 10 times more effective in gene silencing compared to a DNA mutation in the promotor region itself.

Accordingly, measurement of the methylation content of target genes/sequences of interest can facilitate detection for hypermethylation of specific sequences and diagnosis of related disease, determination of disease prognosis, and/or monitoring of disease. If the measurement of methylation can be completed in around 10 minutes, such rapid measurement can facilitate point of care diagnosis, prognosis determination, and disease monitoring. Such measurement of methylation can facilitate other disease monitoring (e.g., in addition to cancer), as long as the disease is correlated with epigenetic alterations like DNA methylation.

Early experimental systems for nanopore based DNA sequencing detected electrical behavior of ssDNA passing through an α-hemolysin (αHL) protein nanopore. Since then, nanopore based nucleic acid sequencing technology has been improved. For instance, solid-state nanopore based nucleic acid sequencing replaces biological/protein based nanopores with solid-state (e.g., semiconductor, metallic gates) nanopores, as described herein.

A nanopore is a small hole (e.g., with a diameter of in the nanometer range that can detect the flow of charged particles (e.g., methylated oligonucleotides, etc.) through the hole by the change in the ionic current and/or tunneling current. Nanopore technology is based on electrical sensing, which is capable of detecting methylation of oligonucleotides in concentrations and volumes much smaller than that required for other conventional detection methods. Advantages of nanopore based methylated oligonucleotide detection include long read length, plug and play capability, and scalability. With advancements in semiconductor manufacturing technologies, solid-state nanopores have become an inexpensive and superior alternative to biological nanopores partly due to the superior mechanical, chemical and thermal characteristics, and compatibility with semiconductor technology allowing the integration with other sensing circuitry and nanodevices.

FIG. 1 schematically depicts a state-of-art solid-state based 2-dimensional (“2D”) nanopore sensing device 100. While, the device 100 is referred to as “two dimensional,” the device 100 has some thickness along the Z axis. In order to address the some of these drawbacks (sensitivity and some of the manufacturing cost) of current state-of-art nanopore technologies, multi-channel nanopore arrays which allows parallel processing of biomolecules may be used to achieve amplification-free and rapid DNA methylation detection. Examples of such multi-channel nanopore arrays are described in U.S. Provisional Patent Application Ser. Nos. 62/566,313 and 62/593,840 and U.S. Utility patent application Ser. No. 16/524,033, the contents of which have been previously incorporated by reference.

As described herein, there is a need for a device (e.g., a bedside/point of care detection system) and method for affordable, rapid, accurate, and early detection of epigenetic alterations in specific genes in a genome. In particular, there is a need for such a device and method for detecting methylation of genomic DNA.

### SUMMARY

Embodiments described herein are directed to nanopore based electrically assisted methylation detection systems and methods of detecting DNA methylation using same. In particular, the embodiments are directed to various types (2D or 3D) of nanopore based methylation detection systems, methods of using nanopore array devices, and methods of methylation detection using same.

In one embodiment, a method of determining an oligonucleotide methylation percentage includes providing a 3D nanopore device having top and bottom chambers, and a 3D nanochannel array disposed in the top and bottom chambers such that the top and bottom chambers are fluidly coupled by a plurality of nanochannels in the 3D nanochannel array. The method also includes purifying an oligonucleotide. The method further includes functionalizing the 3D nanochannel array by coupling an oligonucleotide probe to an inner surface of the 3D nanopore device defining the nanochannel, where the oligonucleotide probe is complementary to the oligonucleotide. Moreover, the method includes adding the purified oligonucleotide to DI water to form an oligonucleotide solution having a known concentration. In addition, the method includes adding the oligonucleotide solution including the oligonucleotide to the top and bottom chambers. The method also includes placing top and bottom electrodes in the top and bottom chambers respectively. The method further includes applying an electrophoretic bias between the top and bottom electrodes. Moreover, the method includes applying a selection bias across first and second gating nanoelectrodes in the 3D nanopore device to direct flow of the oligonucleotide through a nanochannel of the plurality of nanochannels. In addition, the method includes applying a sensing bias through a sensing nanoelectrode in the 3D nanopore device. The method also includes detecting an output current from the sensing nanoelectrode. The method further includes analyzing the output current from the sensing nanoelectrode to determine a methylation percentage of the oligonucleotide.

In one or more embodiments, the method also includes functionalizing the 3D nanochannel array by coupling a second oligonucleotide probe to an inner surface of the 3D nanopore device defining a second nanochannel, where the second oligonucleotide probe is different from the oligonucleotide probe. Analyzing the output current from the sensing electrode to determine a methylation percentage of the oligonucleotide may include comparing the output current and the sensing bias to corresponding values in a reference table for the known concentration. Analyzing the output current from the sensing electrode to determine a methylation percentage of the oligonucleotide may include using an effect of methylation on a charge of a phosphate backbone of the oligonucleotide.

In one or more embodiments, the method also includes applying a second sensing bias through the sensing nanoelectrode in the 3D nanopore device. The method further includes detecting a second output current from the sensing nanoelectrode. Moreover, the method includes analyzing the second output current from the sensing nanoelectrode to determine a second methylation percentage of the oligonucleotide. In addition, the method includes comparing the second methylation percentage of the oligonucleotide to the methylation percentage of the oligonucleotide to confirm the methylation percentage of the oligonucleotide.

In one or more embodiments, the oligonucleotide is an RNA molecule fragment or a DNA molecule fragment. The oligonucleotide may be extracted from cell free DNA, tissue, cell culture medium, serum, urine, plasma, or saliva. Charge carriers in the 3D nanopore device may include the DI water, H+ ions, and OH− ions.

In one or more embodiments, the method also includes removing the oligonucleotide solution including the oligonucleotide from the top and bottom chambers. The method further includes purifying a second oligonucleotide. Moreover, the method includes functionalizing the 3D nanochannel array by coupling a second oligonucleotide probe to an inner surface of the 3D nanopore device defining the nanochannel, where the second oligonucleotide probe is complementary to the second oligonucleotide. In addition, the method includes adding the purified second oligonucleotide to DI water to form a second oligonucleotide solution having a known concentration. The method also includes adding the second oligonucleotide solution including the second oligonucleotide to the top and bottom chambers. The method further includes applying the electrophoretic bias between the top and bottom electrodes. Moreover, the method includes applying the selection bias across the first and second gating nanoelectrodes in the 3D nanopore device to direct flow of the second oligonucleotide through the nanochannel. In addition, the method includes applying the sensing bias through the sensing nanoelectrode in the 3D nanopore device. The method also includes detecting a second output current from the sensing nanoelectrode. The method further includes analyzing the second output current from the sensing nanoelectrode to determine a methylation percentage of the second oligonucleotide.

In one or more embodiments, the method also includes applying a second selection bias across third and fourth gating nanoelectrodes in the 3D nanopore device to direct flow of a second oligonucleotide through a second nanochannel of the plurality of nanochannels. The method further includes applying a second sensing bias through a second sensing nanoelectrode in the 3D nanopore device. Moreover, the method includes detecting a second output current from the second sensing nanoelectrode. In addition, the method includes analyzing the second output current from the second sensing nanoelectrode to determine a methylation percentage of the second oligonucleotide.

In one or more embodiments, analyzing the output current from the sensing electrode to determine a methylation percentage of the oligonucleotide includes differentiating between methyl cytosine methylation and hydroxy methyl cytosine methylation. The method may also include comparing the methylation percentage of the oligonucleotide to a library of methylation patterns corresponding to known mutations to diagnose a disease. The disease may be cancer, atherosclerosis, or aging.

In one or more embodiments, the oligonucleotide probe is a DNA probe, an RNA probe, or a protein probe. The method may also include analyzing the output current from the sensing nanoelectrode to quantify a number of methylation sites in the oligonucleotide. The method may also include applying a rate control bias to a rate control nanoelectrode in the 3D nanopore device to modulate a translocation rate of the oligonucleotide through the nanochannel. The current may be an electrode current or a tunneling current.

In one or more embodiments, the first gating nanoelectrode addresses a first end of the nanochannel, the second gating nanoelectrode addresses a second end of the nanochannel opposite the first end, and a sensing nanoelectrode addresses a first location in the nanochannel between the first and second ends. The method may also include alternatively reversing the electrophoretic bias and the selection bias to direct alternating flow of the oligonucleotides through the nanochannel between the first and second gating nanoelectrodes.

In one or more embodiments, the 3D nanopore device is integrated into a mobile application, a laptop computer, or a desktop computer. The 3D nanopore device may be integrated into microfluidic device, a nanofluidic device, a nanodevice, or a lab-on-chip system. The 3D nanopore device may be integrated into an all-in-one ASIC platform system for extraction and sensing of the oligonucleotide.

In one or more embodiments, the method also includes the 3D nanopore device detecting hybridization of the oligonucleotide to the oligonucleotide probe at a minimum concentration of the oligonucleotide of about 10 femtomolar (limit of detection). The method may also include the 3D nanopore device detecting hybridization of the oligonucleotide to the oligonucleotide probe without amplification of the oligonucleotide or use of PCR. The 3D nanopore device may be integrated into a liquid biopsy panel platform to perform detection without amplification of the oligonucleotide or use of PCR.

In one or more embodiments, the method also includes analyzing the output current from the sensing nanoelectrode to determine a conformation change of the oligonucleotide. The method may also include analyzing the output current from the sensing nanoelectrode to determine a hydration change of the oligonucleotide.

In another embodiment, a method of determining an oligonucleotide conformation change includes providing a 3D nanopore device having top and bottom chambers, and a 3D nanochannel array disposed in the top and bottom chambers such that the top and bottom chambers are fluidly coupled by a plurality of nanochannels in the 3D nanochannel array. The method also includes purifying an oligonucleotide. The method further includes functionalizing the 3D nanochannel array by coupling an oligonucleotide probe to an inner surface of the 3D nanopore device defining the nanochannel, where the oligonucleotide probe is complementary to the oligonucleotide. Moreover, the method includes adding the purified oligonucleotide to DI water to form an oligonucleotide solution having a known concentration. In addition, the method includes adding the oligonucleotide solution including the oligonucleotide to the top and bottom chambers. The method also includes placing top and bottom electrodes in the top and bottom chambers respectively. The method further includes applying an electrophoretic bias between the top and bottom electrodes. Moreover, the method includes applying a selection bias across first and second gating nanoelectrodes in the 3D nanopore device to direct flow of the oligonucleotide through a nanochannel of the plurality of nanochannels. In addition, the method includes applying a sensing bias through a sensing nanoelectrode in the 3D nanopore device. The method also includes detecting an output current from the sensing nanoelectrode. The method further includes analyzing the output current from the sensing nanoelectrode to determine a conformation change of the oligonucleotide.

In still another embodiment, a method of determining an oligonucleotide hydration change includes providing a 3D nanopore device having top and bottom chambers, and a 3D nanochannel array disposed in the top and bottom chambers such that the top and bottom chambers are fluidly coupled by a plurality of nanochannels in the 3D nanochannel array. The method also includes purifying an oligonucleotide. The method further includes functionalizing the 3D nanochannel array by coupling an oligonucleotide probe to an inner surface of the 3D nanopore device defining the nanochannel, where the oligonucleotide probe is complementary to the oligonucleotide. Moreover, the method includes adding the purified oligonucleotide to DI water to form an oligonucleotide solution having a known concentration. In addition, the method includes adding the oligonucleotide solution including the oligonucleotide to the top and bottom chambers. The method also includes placing top and bottom electrodes in the top and bottom chambers respectively. The method further includes applying an electrophoretic bias between the top and bottom electrodes. Moreover, the method includes applying a selection bias across first and second gating nanoelectrodes in the 3D nanopore device to direct flow of the oligonucleotide through a nanochannel of the plurality of nanochannels. In addition, the method includes applying a sensing bias through a sensing nanoelectrode in the 3D nanopore device. The method also includes detecting an output current from the sensing nanoelectrode. The method further includes analyzing the output current from the sensing nanoelectrode to determine a hydration change of the oligonucleotide.

The aforementioned and other embodiments of the invention are described in the Detailed Description which follows.

### BRIEF DESCRIPTION OF THE DRAWINGS

The drawings described below are for illustration purposes only. The drawings are not intended to limit the scope of the present disclosure. The drawings illustrate the design and utility of various embodiments of the present disclosure. It should be noted that the figures are not drawn to scale and that elements of similar structures or functions are represented by like reference numerals throughout the figures. In order to better appreciate how to obtain the recited and other advantages and objects of various embodiments of the disclosure, a more detailed description of the present disclosure will be rendered by reference to specific embodiments thereof, which are illustrated in the accompanying drawings. Understanding that these drawings depict only typical embodiments of the disclosure and are not therefore to be considered limiting of its scope, the disclosure will be described and explained with additional specificity and detail through the use of the accompanying drawings.

FIG. 1 schematically illustrates a prior art solid-state 2D nanopore device;

FIGS. 2 to 4 schematically illustrate 3D nanopore devices according to various embodiments.

FIGS. 5 to 11 schematically depict a method for detecting DNA methylation using a 3D nanopore device according to some embodiments.

FIGS. 12A and 12B schematically depict a method for manufacture a nanopore device according to some embodiments.

FIG. 13 is a 3D histogram illustrating a relationship between a percentage of DNA methylation and an output current in a nanopore methylation detection device according to some embodiments.

FIG. 14 is a flow-chart depicting a method of detecting methylation of oligonucleotides using a nanopore detection system according to some embodiments.

FIG. 15 schematically depicts a mechanism of detecting/classifying methylation of DNA in a 3D nanopore device/sensor according to some embodiments.

FIGS. 16-18 schematically illustrate conformational changes of double stranded DNA inside a 3D nanopore device/sensor according to some embodiments.

FIG. 19 schematically illustrates a hydration mediated mechanism of signal change in double stranded DNA inside a 3D nanopore device/sensor according to some embodiments.

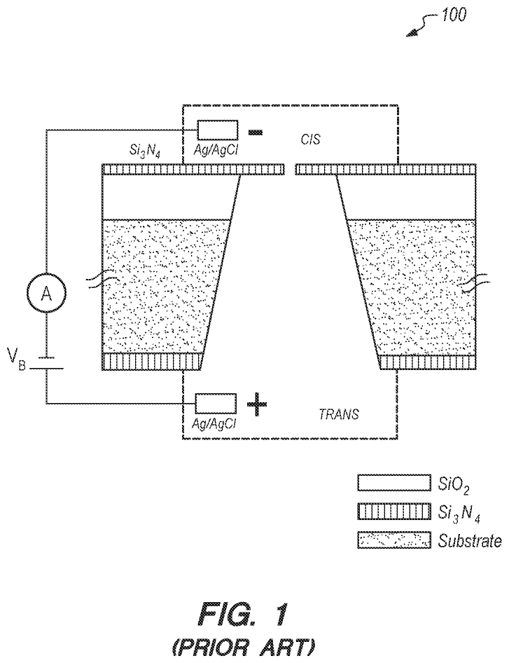

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

Methods are described herein to achieve amplification-free and rapid detection of DNA methylation (e.g., in less than 10 minutes). Nanopore electrically assisted DNA methylation detection devices that efficiently and effectively detect DNA methylation by manipulating potentials to increase hybridization of DNA and detecting electrical characteristics generated by hybridization of methylated DNA are described herein. Such detection devices and methods can be used in various biomolecular arrays, including microarrays, CMOS arrays, and nanopore arrays (e.g., solid-state, and hybrid nanopore arrays). Such detection devices and methods can also be used with various multi-channel nanopore arrays, including the 3D multi-channel nanopore arrays described above and planar multi-channel nanopore arrays.

Multi-channel nanopore arrays that allow parallel processing of DNA methylation detection may be used to achieve amplification-free and rapid methylation detection. Examples of such multi-channel nanopore arrays are described in U.S. Provisional Patent Application Ser. Nos. 62/566,313 and 62/593,840, and U.S. Utility patent application Ser. No. 16/524,033, the contents of which have been previously incorporated by reference. Such multi-channel nanopore arrays can be electrically addressed to direct charged particles (e.g., methylated DNA) to specific channels in these multi-channel nanopore arrays. Other arrays are coupled to microfluidic channels outside the array. Electrically addressing and sensing individual nanopore channels within multi-channel nanopore arrays, as described in U.S. Provisional Patent Application Ser. No. 62/612,534, the contents of which have been previously incorporated by reference, can facilitate more efficient and effective use of multi-channel nanopore arrays to achieve low cost, high throughput, amplification-free, and rapid detection of methylated DNA.

### Mechanism of Characterization

In some embodiments, the mechanism of characterization (e.g., sensing mechanism) of methylation patterns leverages certain properties of oligonucleotide bases (e.g., in DNA molecules). The guanine base is one of the four base pairs in the DNA molecules, which is easily oxidized. In terms of energy levels, the electrical charge of a guanine base is just 0.2 eV. Therefore the electrical charge of the guanine base can migrate easily along a DNA chain into the next oxidizing group or the next guanosine. The electrical charges/energies of guanine-cytosine (“G-C”) and adenine-thymine (“A-T”) base pairs in DNA function as relative charge carriers, allowing an electrical charge (e.g., of a guanine base) to hop along the length of the DNA molecule between charge carriers. A positively charged “hole” in a DNA molecule may have a lower energy at one or more G-C sites and this hole may move from one G-C pair to the next by coherent tunneling through the A-T sites in the DNA molecule. As such, one or more positively charged holes in a DNA molecule can affect (e.g., reduce the negative charge of) the charge of the entire DNA molecule.

The mechanism of characterization (e.g., sensing mechanism) of methylation patterns may also leverage hydration effects on electrical fields of DNA molecules. In some embodiments, the mechanism of characterization (e.g., sensing mechanism) is performing in a de-ionized (“DI”) water solution of oligonucleotides (e.g., a DNA strand) such that water molecules and oligonucleotides form hydrated bio-interfaces that effect electrical charge characteristics. The hydrophobicity of DNA base pairs and the DNA double helix results in a structure that positions the hydrophobic DNA base pairs away from the water in a DI water solution. The negatively charged backbone of the DNA strand attracts positively charged ions around the backbone. Methylation adds a methyl group (e.g., to cytosine), resulting in an almost neutral energy level that can cover the negative charge of the DNA backbone. Further, water molecules in a DI water solution can form a water shell around the DNA strand in the hydration state.

The mechanism of characterization (e.g., sensing mechanism) of methylation patterns may also leverage charge effects of hydration of DNA molecules in CpG islands. When hydrogen atoms (e.g., from water molecules) face the phosphate backbone of the DNA, they affect each other. The neutral nature of methyl groups added during methylation, and their interface with the water molecules result in a DNA backbone covered by hydrogen atoms. Accordingly, these methylation mediated interactions can be detected by their effect on the charge of the DNA molecule, which can be sensed by imbedded electrodes. The 3-dimensional (“3D”) sensors described herein and methods using same are capable of sensing the charge changes in the reaction chamber, which include the total charge of the DNA molecules in solution.

Methylation of DNA (e.g., cytosine in C-G pairs) also affects the stiffness of the methylated dinucleotides (e.g., deformational mode dependent effects). Methylation increases the stiffness of the dinucleotides marginally, but increases the stiffness of the neighboring dinucleotides more significantly. Stiffening is further enhanced for consecutively methylated dinucleotides, which may result in the effect of hypermethylation. Steric interactions between the added methyl groups and the nonpolar groups of the neighboring nucleotides may be responsible for the stiffening in many embodiments. Hydration maps show that methylation also alters the surface hydration structure in various ways. Resistance to deformation of methylated DNA may contribute to the stiffening of DNA for deformational modes lacking steric interactions. The effect of methylation on the conformational behavior of DNA may depend on the local sequence around the methylation site.

Some embodiments of mechanisms of characterization (e.g., sensing mechanism) of methylation patterns described herein are based at least partially on DNA hydration and the methyl group neutralization of the DNA backbone, which may affect the H+ and OH− groups in the reaction chamber. Some 3D nanopore sensor arrays described herein facilitate detection of methylation with increased sensitivity and reduced detection limit by decreasing the Debby lenses of the sensing areas in the nanochannels.

One exemplary approach for measuring or sensing the DNA is to analyze fluctuations in helicoidal parameters as indicated by electrical signals measured by imbedded electrodes inside a 3D nanopore sensor arrays, as described herein, DNA conformation change is one of the mechanisms that can alter the charge in the electrode area and generate a signal. DNA methylation results in weak fluctuations in the DNA structure resulting in stiffer DNA. Also, methylation adds methyl groups that change the hydration environment of the DNA molecule adjacent the methylation site. These various mechanisms reduce the solving energy for better characterization of hydration shells around the methyl group.

Because water has a high affinity for hydroxymethylcyosine (“hmC”), G-hmC base pairs experience the largest charge fluctuations. In contrast, water is less apt to solvate the hydrophobic methyl group of methylcytosine (“mC”), which increases the rigidity/inflexibility of G-mC base pairs. Methylation of cytosines in the substitute sequences poly(deoxyguanylic-deoxycytidylic) acid sodium salt (“poly(dG-dC)”)⋅permits the development of Z-DNA at more vulnerable ionic quality than is required for unmethylated DNA.

In other embodiments, the output current mean values vary according to the pattern: hmC<C<mC. Consequently, there may be differences in DNA adaptability to methylation.

### Exemplary Nanopore Devices

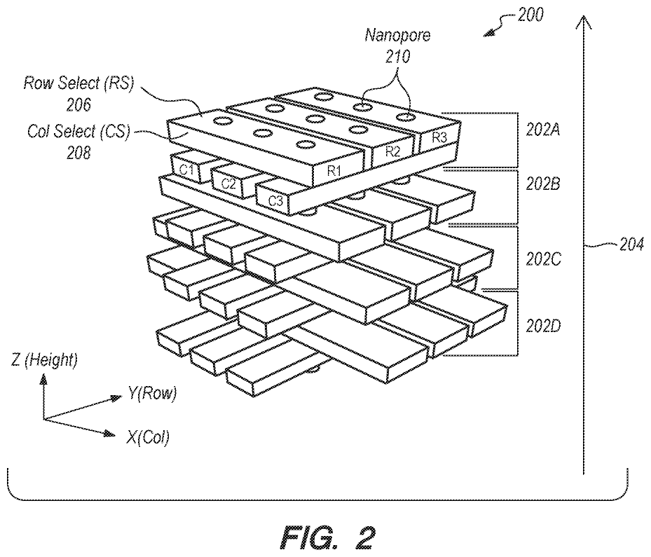

FIG. 2 schematically depicts a nanopore device 200 with a three dimensional (“3D”) array architecture according to one embodiment. The device 200 includes a plurality of 2D arrays or layers 202A-202D stacked along a Z axis 204. While the 2D arrays 202A-202D are referred to as “two dimensional,” each of the 2D arrays 202A-202D has some thickness along the Z axis.

The top 2D array 202A includes first and second selecting (inhibitory nanoelectrode) layers 206, 208 configured to direct movement of charged particles (e.g., biopolymers) through the nanopores 210 (pillars, nanochannels) formed in the first and second selecting layers 206, 208. The first selecting layer 206 is configured to select from a plurality of rows (R1-R3) in the 2D array 202A. The second selecting layer 208 is configured to select from a plurality of columns (C1-C3) in the 2D array 202A. In one embodiment, the first and second selecting layers 206, 208 select from the rows and columns, respectively, by modifying a charge adjacent the selected row and column and/or adjacent to the non-selected rows and columns. The other 2D arrays 202B-202D include rate control/current sensing nanoelectrodes. Rate control/sensing nanoelectrodes may be made of highly conductive metals and polysilicon, such as Au—Cr, TiN, TaN, Ta, Pt, Cr, Graphene, Al—Cu, etc. The rate control/sensing nanoelectrodes may have a thickness of about 0.3 to about 1000 nm. Rate control/sensing nanoelectrodes may also be made in the biological layer in hybrid nanopores. Each sensing nanoelectrode may be operatively coupled/address to a nanopore 210 pillar, such that each nanopore 210 pillar may be operatively coupled to a particular memory cell. Electrical addressing in nanopore devices is described in U.S. Provisional Patent Application Ser. No. 62/612,534, the contents of which have been previously incorporated by reference.

Hybrid nanopores include a stable biological/biochemical component with solid-state components to form a semi-synthetic membrane porin to enhance stability of the nanopore. For instance, the biological component may be an αHL molecule. The αHL molecule may be inserted into a SiN based 3D nanopore. The αHL molecule may be induced to take on a structure to ensure alignment of the αHL molecule with the SiN based 3D nanopore by apply a bias to a nanoelectrode (e.g., in the top 2D array 202A).

The nanopore device 200 has a 3D vertical pillar stack array structure that provides a much larger surface area for charge detection than that of a conventional nanopore device having a planar structure. As a charged particle (e.g., biopolymer) passes through each 2D array 202A-202E in the device, its charge can be detected with a detector (e.g., nanoelectrode) in some of the 2D arrays 202B-202E. Therefore, the 3D array structure of the device 200 facilitates higher sensitivity, which can compensate for a low signal detector/nanoelectrode. The integration of memory cells into the 3D array structure minimizes any memory related performance limitations (e.g., with external memory device). Further, the highly integrated small form factor 3D structure provides a high density nanopore array while minimizing manufacturing cost.

In use, the nanopore device 200 is disposed between and separating top and bottom chambers (not shown) such that the top and bottom chambers are fluidly coupled by the nanopore pillars 210. The top and bottom chambers include a nanoelectrode (e.g., Ag/AgCl2, etc.) and a buffer (electrolyte solutions or DI water with KCl) containing the charged particles (e.g., DNA) to be detected. Different nanoelectrodes and electrolyte solutions can be used for the detection of different charged particles.

Electrophoretic charged particle translocation can be driven by applying a bias to nanoelectrodes disposed in a top chamber (not shown) adjacent the top 2D array 202A of the nanopore device 200 and a bottom chamber (not shown) adjacent the bottom 2D array 202E of the nanopore device 200. In some embodiments, the nanopore device 200 is disposed in a between top and bottom chambers (not shown) such that the top and bottom chambers are fluidly and electrically coupled by the nanopore pillars 210 in the nanopore device 200. The top and bottom chambers may contain the electrolyte solution.

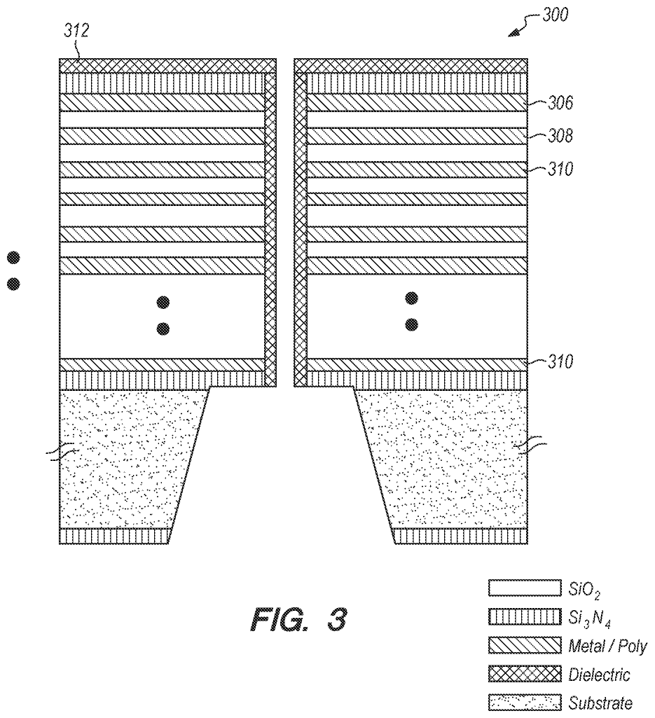

FIG. 3 schematically depicts a nanopore device 300 according to one embodiment. The nanopore device 300 includes an insulating membrane layer (Si3N4) followed by row and column select (inhibitory nanoelectrodes) 306 and 308, respectively (e.g., metal or doped polysilicon), and a plurality (1st to Nth) of cell nanoelectrodes 310 (e.g., metal or doped polysilicon). The nanoelectrodes 306, 308, 310 of the nanopore device 300 are covered by an insulator dielectric film 312 (e.g., Al₂O₃, HfO₂, SiO₂, ZnO).

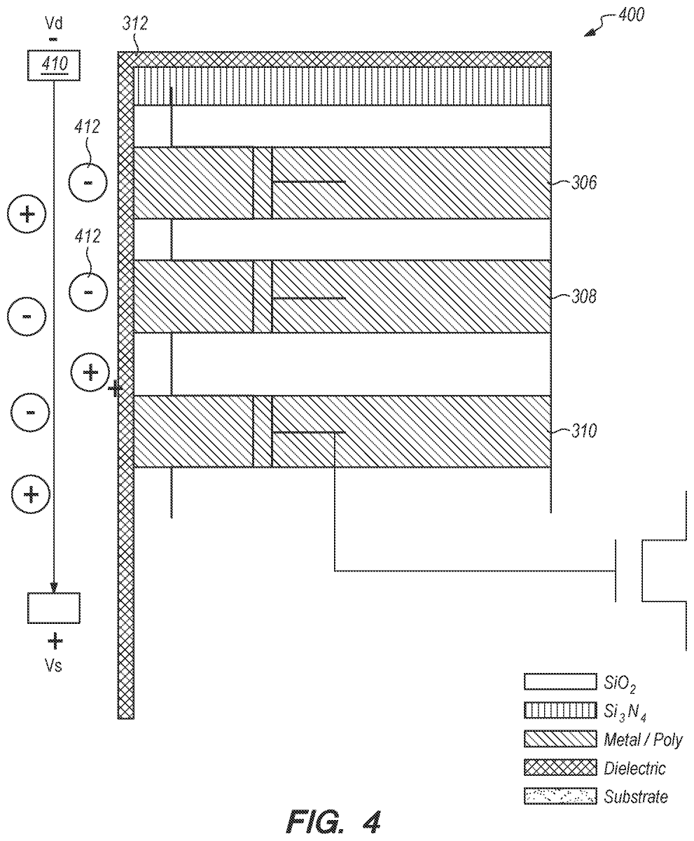

As shown in FIG. 4, when a translocation rate control bias signal 410 for column and row voltages (e.g., Vd) is applied to the 3D nanopore sensor array 400, row and column inhibitory voltage/bias pulses are followed by a verify (sensing) voltage/bias pulse (e.g., Vg1, Vg2), as described herein. Vg3 and following electrodes (Vg4˜VgN) are sensing and translocation electrodes. An exemplary signal 410 is depicted in FIG. 4 overlaid on top of the 3D nanopore sensor array 400. Inhibitory biases are applied to deselect various column and row nanopore pillar channels/nanochannels, respectively. During sensing operation, both column and row (inhibitory) select nanoelectrodes are selected. The resulting surface charge 412 can be detected as a change in an electrical characteristic, such as current.

In some embodiments, the nanoelectrodes can detect current modulations using a variety of principles, including ion blockade, tunneling, capacitive sensing, piezoelectric, and microwave-sensing. It is also possible that ionic concentration or so called ionic current change in the electrode (detected by the reference electrode) can be amplified and accurately sensed by the attached CMOS transistor as shown in the FIG. 4.

### Exemplary Nanopore Electrically Assisted DNA Methylation Detection Device and Method

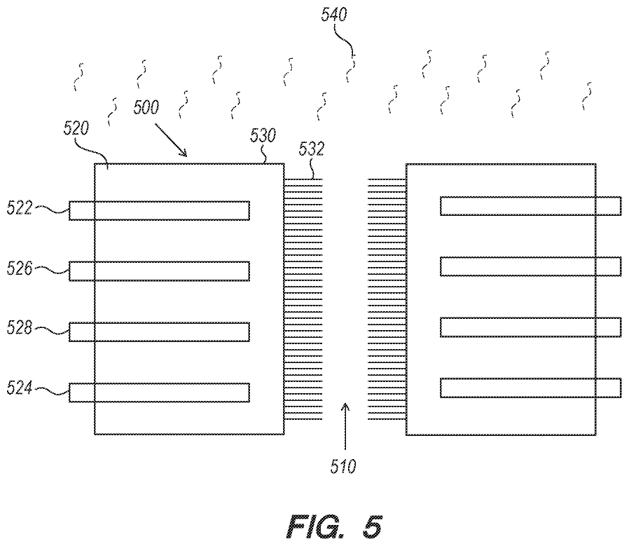

FIG. 5 depicts a nanopore electrically assisted DNA methylation detection device according to some embodiments. While a portion of a nanopore detection device 500 including a single nanochannel 510 is depicted in FIG. 5, nanopore electrically assisted DNA methylation (e.g., epigenetic change) detection devices can include a 3D array having a plurality of nanochannels. DNA methylation sensing structure such as the nanopore detection device 500 depicted in FIG. 5 leverage the charge sensitivity of the nanochannels and the large surface area resulting from parallel processing and 3D arrays to facilitate rapid amplification-free detection of DNA methylation.

The nanopore detection device 500 includes nanoelectrodes 522, 524, 526, 528. These nanoelectrodes 522, 524, 526, 528 are independently electrically addressed to control flow through the nanochannel 510 (first and second gating nanoelectrodes 522, 524) and detect charges in the nanochannel 510 (first and second sensing nanoelectrodes 526, 528).

The nanopore detection device 500 also includes probes (PNA, DNA morpholino oligomers) 532 that are coupled to an interior surface 530 of the nanochannel 510. The interior surface 530 can include Al₂O₃. The Al₂O₃ includes a large number of hydroxyl groups to facilitate functionalization for immobilization of probes 532 on the interior surface 530 of the nanochannel 510. The probes 532 can be generated using known molecular biology techniques to be complementary to the target region within genomic DNA (e.g., CpG islands in a promoter region). The probes (e.g., DNA, RNA, PNA, LNA, Morpholinos, etc.) 532 can have a variety of lengths (e.g., 24 base pairs, 40 base pairs, etc.)

The probes 532 can be coupled/covalently bonded to the interior surface using vapor-phase silanization. The thickness of the organic coating of probes 532 can also be modulated by modifying the time of the vapor-phase silanization.

In some embodiments, the nanopore device is first treated with O₂ plasma to generate —OH groups on the oxide dielectric (Al₂O₃, HfO₂, etc.) Al₂O₃ substrate thereby activating the substrate for attaching target functional groups. Then, 3-aminopropyl triethoxy silane (APTES) is used for silanization because it is effective on a variety of possible surface structures and because it is extremely reactive. Before covalent attachment of the probes 532, the nanopore device 510 is exposed to silanes (e.g., APTES And OTMS 1:3 ratio in ethanol) in vapor phase by placing it in a dynamically pumped low vacuum chamber adjacent a glass holder containing 50 μl of APTES (from Sigma-Aldrich), at ambient temperature and a base pressure of about 30 kPa. Then, the nanopore device 510 is removed from the vacuum chamber and immersed in a 2.5% glutaraldehyde solution (Sigma-Aldrich) for one hour. Next the nanopore device 510 is removed from the cross-linker and washed twice in IPI and twice in double distilled water. Finally, the nanopore device 510 is treated (e.g., by immersion) overnight at 37° C. with a 100 nM amino-modified probe. After each step, the nanopore device is washed in Ultrapure DNase/RNase-Free Distilled water (used as washing buffer). Using such methods, covalent attachment/immobilization of the probes 532 can be accomplished in approximately 24 hours, or in eight hours at 45° C.

The sensitivity of the nanopore detection device 500 hybridization of electrically target biomolecules 540 (e.g., methylated oligonucleotides) to the probes 532 covalently bonded to the interior surface 530 of the nanochannel 510 is such that a single base mismatch can be detected based on the resulting difference in electrical charge. The parallel processing resulting from the 3D array structure of nanopore devices dramatically increases the interface area between the nanopore devices and the methylated oligonucleotides to be detected, thereby increasing sensitivity to a level sufficient for a point of care diagnosis and determination of prognosis of a variety of disorders (e.g., genetic disorders).

The first and second gating nanoelectrodes 522, 524 are independently addressed and can therefore be rapidly electrically modified to generate a “ping-pong” movement of target biomolecules 540 that increases hybridization of the target biomolecules 540 and the probes 532. A potential across the first and second gating nanoelectrodes 522, 524 in the nanochannel 510 can be rapidly reversed by applying current to the first and second gating nanoelectrodes 522, 524. The first and second gating nanoelectrodes 522, 524 can also be addressed to control translocation of target biomolecules 540 through the nanochannel 510.

The target charge biomolecules 540 can be many varieties of nucleic acids such as DNA, cDNA, mRNA, etc. The probes 532 can be complementary DNA strands, locked nucleic acid (LNA) oligomers, neutral backbone oligomers like peptide nucleic acids (PNA), DNA morpholino oligomers, or any type of complementary strands that can hybridize with the target charge biomolecules 540.

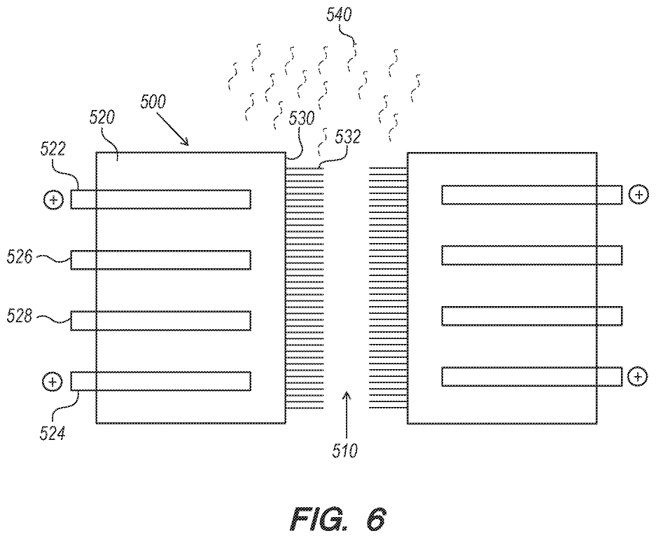

As shown in FIG. 5, before any current/potential is applied to the nanopore detection device 500, the target biomolecules 540 are not attracted to the nanochannel 510. FIG. 6 depicts application of current to generate a positive potential in the first and second gate nanoelectrodes 522, 524. This positive potential attracts the negatively target biomolecules 540 toward the nanochannel 510.

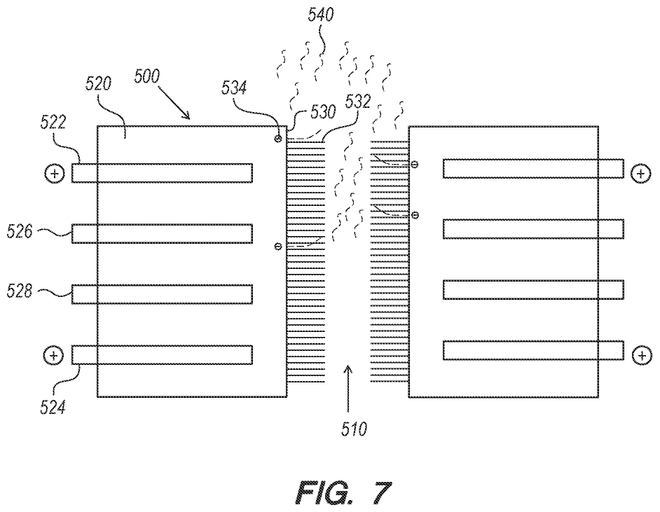

FIG. 7 depicts continued application of current to generate a positive potential in the first and second gate nanoelectrodes 522, 524. Over time, some of the negatively target biomolecules 540 enter the nanochannel 510, and interact with the probes 532 covalently bonded to the interior surface 530 of the nanochannel 510. This interaction between the negatively target biomolecules 540 and the probes 532 results in hybridization between the two molecules. This electrically connects the negatively target biomolecules 540 to the first and second sensing nanoelectrodes 526, 528, which can detect the negative charges 534 associated with the negatively target biomolecules 540.

FIG. 5 depicts a modification of the electrical potentials in the first and second gate nanoelectrodes 522, 524. In FIG. 5, current is no longer applied to the first gate nanoelectrode 522, eliminating the positive potential therein. However, current is maintained across the second gate nanoelectrode 524 to maintain a positive potential therein. This change in potential draws the negatively target biomolecules 540 in the nanochannel 510 toward the second gate nanoelectrode 524, as indicated by the flow arrow 550. FIG. 5 also shows that more negatively target biomolecules 540 have hybridized to the probes 532 in the nanochannel 510.

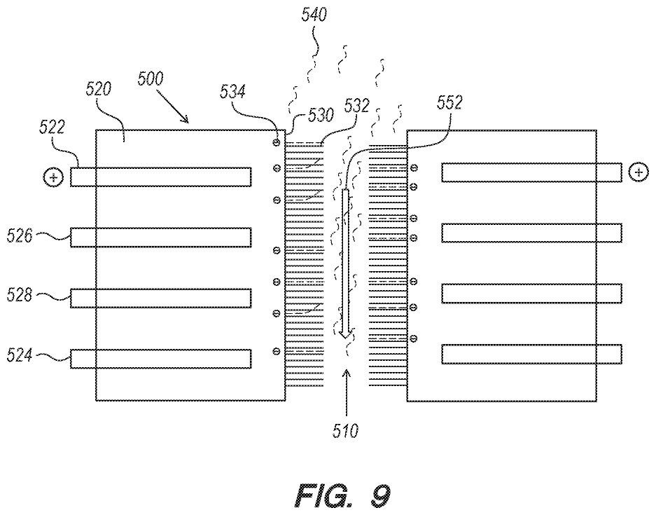

FIG. 9 depicts another modification of the electrical potentials in the first and second gate nanoelectrodes 522, 524. In FIG. 9, current is no longer applied to the second gate nanoelectrode 524, eliminating the positive potential therein. However, current is applied across the first gate nanoelectrode 522 to maintain a positive potential therein. This change in potential draws the negatively target biomolecules 540 in the nanochannel 510 back toward the first gate nanoelectrode 522, as indicated by the flow arrow 552. FIG. 9 also shows that, with more exposure of the charge biomolecules 540 to the probes 532 in the nanochannel 510, even more negatively target biomolecules 540 have hybridized to the probes 532.

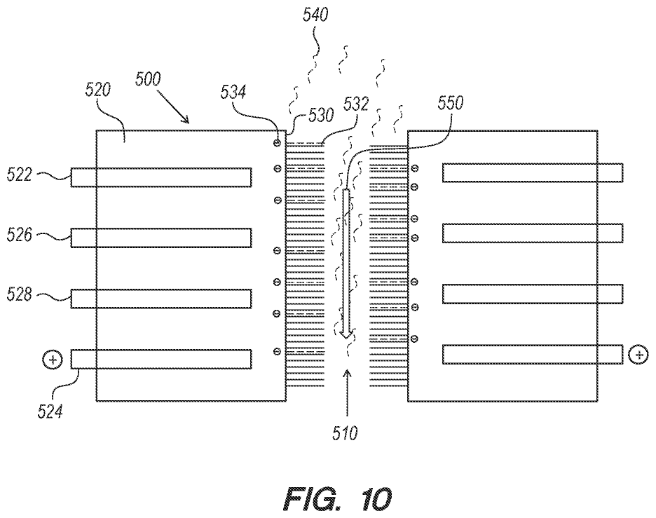

FIG. 10 depicts still another modification of the electrical potentials in the first and second gate nanoelectrodes 522, 524. In FIG. 9, current is no longer applied to the first gate nanoelectrode 522, eliminating the positive potential therein. However, current is applied across the second gate nanoelectrode 524 to maintain a positive potential therein. This change in potential draws the negatively target biomolecules 540 in the nanochannel 510 back toward the second gate nanoelectrode 524, as indicated by the flow arrow 550. FIG. 10 also shows that, with even more exposure of the charge biomolecules 540 to the probes 532 in the nanochannel 510, still more negatively target biomolecules 540 have hybridized to the probes 532.

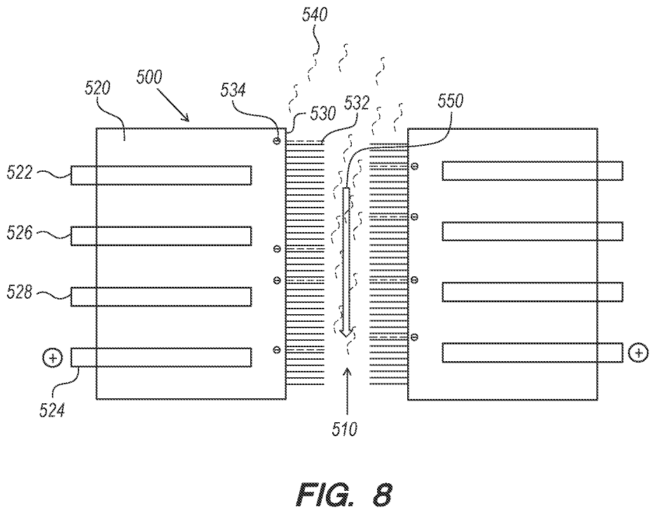

The direction changes depicted in the flow arrows 550, 552 in FIGS. 5 to 10 depict the first two direction changes in the “ping-pong” movement of target biomolecules 540 that increases hybridization of the target biomolecules 540 and the probes 532. The direction changes are controlled by changing the electrical potentials in the first and second gate nanoelectrodes 522, 524, which is in turn modified by alternating the current applied thereto. Because currents can be applied to the individually electrically addressed first and second gate nanoelectrodes 522, 524 under processor control, the alternation of current and electrical potentials can be executed rapidly. The “ping-pong” movement of charged biomolecules 540 increases the amount of time the charged biomolecules 540 are exposed to the probes 532 in the nanochannel 510, thereby increasing the amount of hybridization between the two molecules. While only one or two changes of direction are depicted in FIGS. 5 to 10, a biomolecule detection method can include many more changes of direction to increase the hybridization of the target biomolecules 540.

FIG. 11 depicts the end of a series of “ping-pong” movements in a biomolecule detection method. At the end of the detection method, a plurality of negatively target biomolecules 540 (methylated oligonucleotide) have hybridized to the probes 532, which are themselves covalently bonded to the interior surface 530 of the nanochannel 510. As each negatively target biomolecules 540 hybridizes to a probe 532, its additional negative charge 534 is detected by the first and/or second sensing nanoelectrode 526, 528. The sensing nanoelectrodes 526, 528 are sufficiently sensitive to distinguish single base pair mismatches. Therefore, the sensing nanoelectrodes 524, 528 can detect the negative charges 534 associated with hybridization of each target biomolecules 540. As such, the nanopore detection device 500 can rapidly (e.g., under 10 minutes) detect and quantitate target DNA methylation in a solution.

While the nanopore detection device 500 depicted in FIGS. 5 to 11 is configured to detect only a single negatively charged target biomolecules 540 during a particular procedure, nanopore detection devices according to other embodiments can be configured to detect multiple negatively charged target biomolecules (e.g., methylated oligonucleotides). Such nanopore detection devices include a plurality of probes that (1) hybridized with different negatively charged target biomolecules and (2) have different lengths. Because the probes have different lengths, hybridization of different negatively charged target biomolecules will result in a different amount of negative charge being electrically added to the interior surface of the nanochannel. The sensing nanoelectrodes are sufficiently sensitive to distinguish these different amounts of negative charge, and thereby distinguish hybridization of different negatively charged target biomolecules.

### Exemplary Nanopore Device Manufacturing Method

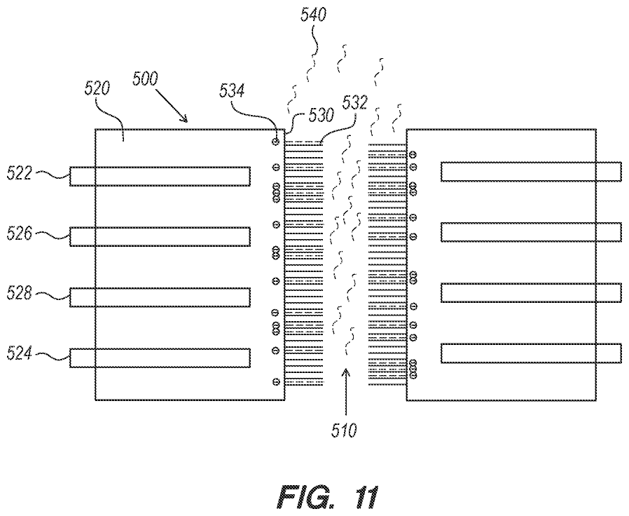

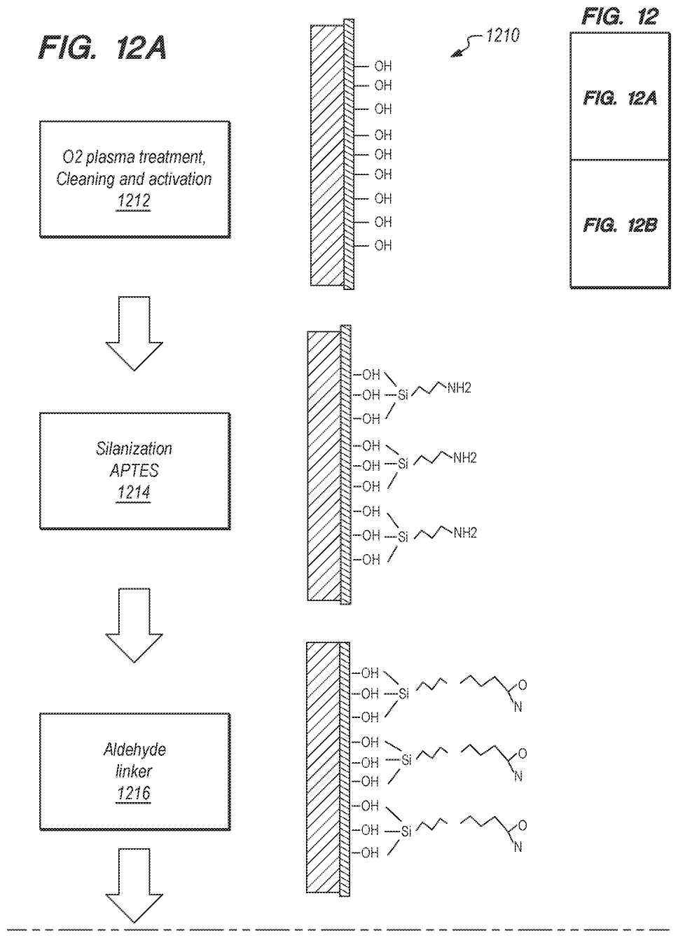

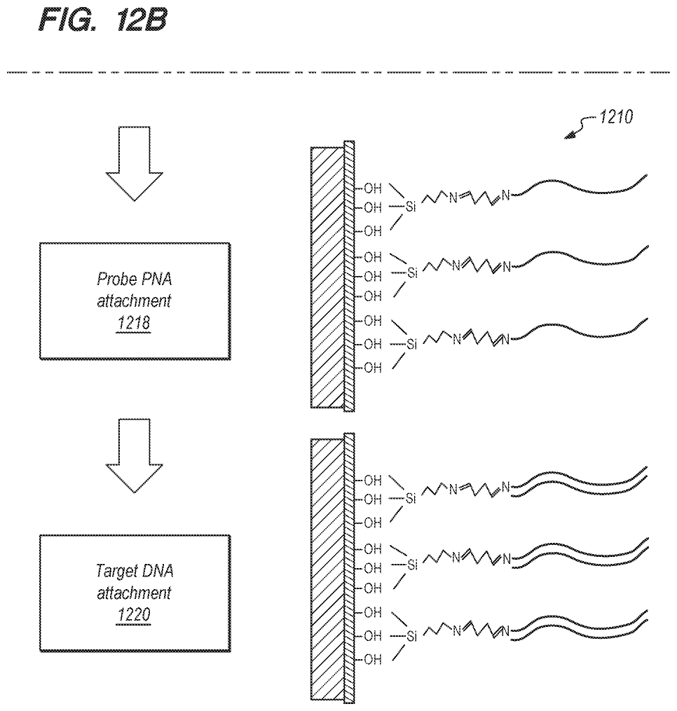

FIGS. 12A and 12B schematically depict a method 1210 for manufacture a nanopore device, such as the nanopore detection devices 500, 600 described above, according to some embodiments.

At step 1212, an interior surface of the nanopore device (in the nanochannel) is O₂ plasma treated, cleaned, and activated. At step 1214, the surface of the device is silanized by treating with (3-aminopropyl)triethoxysilane (APTES) to functionalize the surface. At step 1216, an aldehyde linker is attached to the functionalized surface. At step 1218 (FIG. 12B), a probe (e.g., PNA) is attached to the surface via the aldehyde. At step 1220, the negatively charged target biomolecule (e.g., methylated DNA) attaches to the probe on the surface and changes the charge of the surface for electrically detecting the negatively charged target biomolecule, as described above.

### Methylation Effect on Output Current

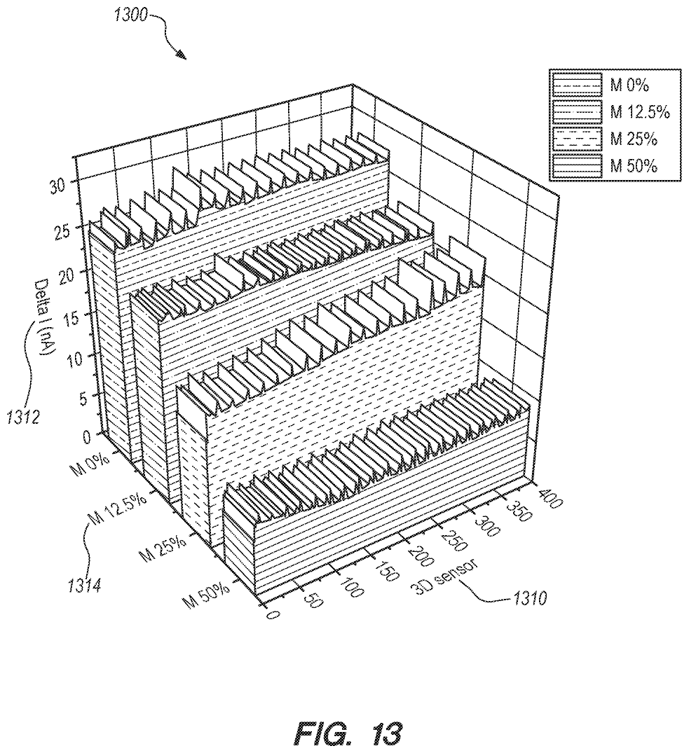

FIG. 13 is a 3D histogram 1300 showing measured output current 1312 vs. applied sensing bias 1310 for a variety of methylation percentages 1314 (for an oligonucleotide complementary to an oligonucleotide probe). Five types of control DNA samples containing different percentage of the methylation 0%, 12.5%, 25%, 50% and 100% 1314 were prepared and complementary probes were designed. After simple functionalization with APTES, a glutaraldehyde linker was added, and the probes were incubated into particular locations in the 3D nanopore sensor arrays. Real time measurements of output currents 1312 for different concentrations of DNA methylation 1314 were performed at a variety of sensing biases 1310 and the results summarized in FIG. 13. As shown in FIG. 13, as the percentage of methylation 1314 increases, the signal/output current decreases 1312 (e.g., due to neutralization of the negative backbone of the DNA and water methyl interaction).

### Blocking Electron Transfer

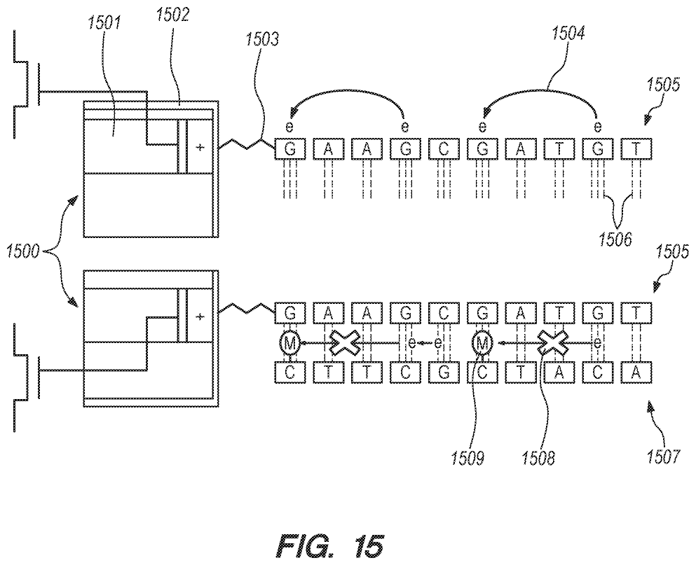

FIG. 15 schematically depicts the mechanism of the detecting/classifying methylation of DNA in a 3D nanopore device/sensor 1500 according to some embodiments. 1501 represents a gate electrode, 1502 represents a dielectric layer with silane. 1503 represents a bond between a designed oligonucleotide probe strand 1505 and a surface of the 3D nanopore device/sensor 1500. 1504 represents an electron transfer between guanine bases. 1506 represents the different hydrogen bounding between A-T and G-C base pairs. 1507 represents a target sequence from a clinical sample, which carries methyl groups. The target sequence/oligonucleotide strand 1507, which has been methylated to a certain degree, is complementary to the oligonucleotide probe strand 1505, and therefore bonds thereto; As shown at 1508, the electron pathway from base to base is blocked by a methyl group 1509 (e.g., in methyl cytosine). This blockage reduces the output current measured by the gate electrode 1501 of the 3D nanopore device/sensor 1500. The amount of reduction is related to the percentage of methylation of the target sequence 1507 (as shown in FIG. 13).

The top embodiment in FIG. 15 illustrates that, when a positive gate bias is applied to the gate electrode 1501 in the 3D nanopore device/sensor 1500, electrons in the oligonucleotide probe 1505, which is attached to the surface of the device 1500, migrate to the gate electrode 1501. The electrons migrate 1504 between the most easily oxidized sites in the DNA strand 1505, which are guanine bases. The electrons continue to migrate to the next easily oxidized base through the DNA strand 1505, which is next guanine base, until it reaches the gate electrode 1501, which the electrons are sensed (e.g., as an output current).

The bottom embodiment in FIG. 15 illustrates that, when a target sequence/oligonucleotide strand 1507 is added to the 3D nanopore device/sensor 1500, the target oligonucleotide strand 1507 bonds to the oligonucleotide probe 1505. After attachment of the target oligonucleotide strand 1507, when a positive gate bias is applied to the gate electrode 1501, the methyl groups 1509 in the methylated cytosine based interrupts the electron transfer mechanism, reducing electron transfer and signal depending on the percentage of methylation of the target oligonucleotide strand 1507. The measured electrical signal (e.g., output current) can be compared with reference methylation percentage profiles (see FIG. 13) to determine the methylation pattern of target oligonucleotide strand 1507.

### Conformational Changes

In some embodiments, methylated and un-methylated oligonucleotides have different conformations, with methylation resulting in a conformation change. The different conformation of a methylated oligonucleotide may change the charge signal at the surface of a 3D nanopore device/sensor electrode. The change in surface charge signal may result in changes in the signal read by the electrode (e.g., output current). The measured changes in the signal may be analyzed to determine conformational changes.

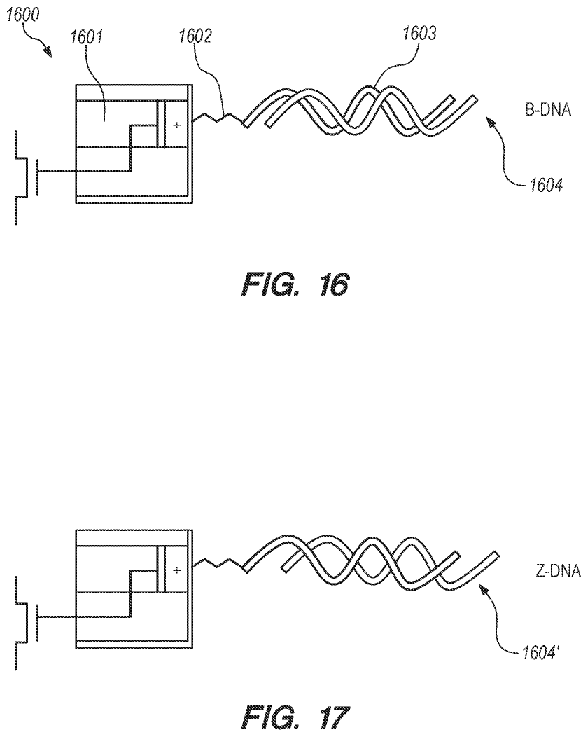

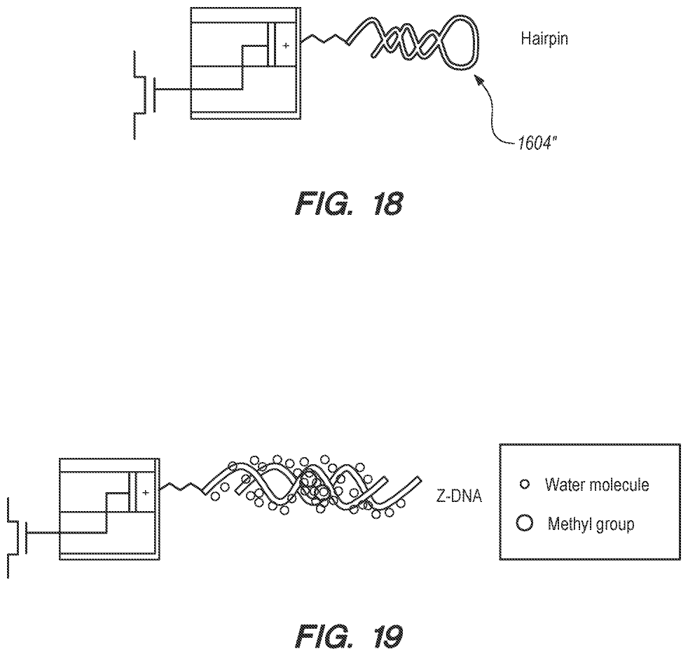

FIGS. 16-18 schematically illustrate conformational changes of the double stranded DNA inside a 3D nanopore device/sensor 1600 according to some embodiments. 1601 represents an electrode (e.g., gate or sensing electrode) and surface structures of the device 1600, 1602 represents a dielectric layer with silane. 1602 represents a bonding site between a designed oligonucleotide probe strand 1603 and a surface of the 3D nanopore device/sensor 1600. 1604 represents a target sequence/oligonucleotide strand. DNA conformation/configuration can change based on the environment of the DNA molecule. For instance, various ions can change the DNA conformation/configuration into a different form of the configuration. FIG. 16 shows the target sequence/oligonucleotide strand 1604 in a B-DNA configuration. FIG. 17 shows the target sequence/oligonucleotide strand 1604′ in a Z-DNA configuration. FIG. 18 shows the target sequence/oligonucleotide strand 1604″ in a “hairpin” configuration. The 3D nanopore device/sensor 1600 can measure signal changes when the target sequence/oligonucleotide strand 1604, 1604′, 1604″ bonds to an oligonucleotide probe 1603 in a DI water environment. These real time signal changes may be analyzed to determine conformational changes.

### Hydration Changes

In some embodiments, methylation may result in changes to hydration of the oligonucleotide. Hydration changes may affect the sensing mechanism by changing the oligonucleotide configuration during hydrogen binding between the complimentary strands. The configuration change may result in changes in the signal read by the electrode (e.g., output current). The measured changes in the signal may be analyzed to determine hydration changes.

FIG. 19 schematically illustrates the hydration mediated mechanism of signal change in DNA molecules with methylated cytosine bases according to some embodiments. Methylated cytosine bases affect the extent of hydration of the target sequence/oligonucleotide strand. The hydration changes, in turn, affect the charge arrangements in the sequence/oligonucleotide strand and the oligonucleotide probe. The 3D nanopore device/sensor can measure signal changes when the target sequence/oligonucleotide strand bonds to an oligonucleotide probe in a DI water environment. These real time signal changes may be analyzed to determine hydration changes.

### Method of Detecting Methylation of DNA Using Nanopore Detection System

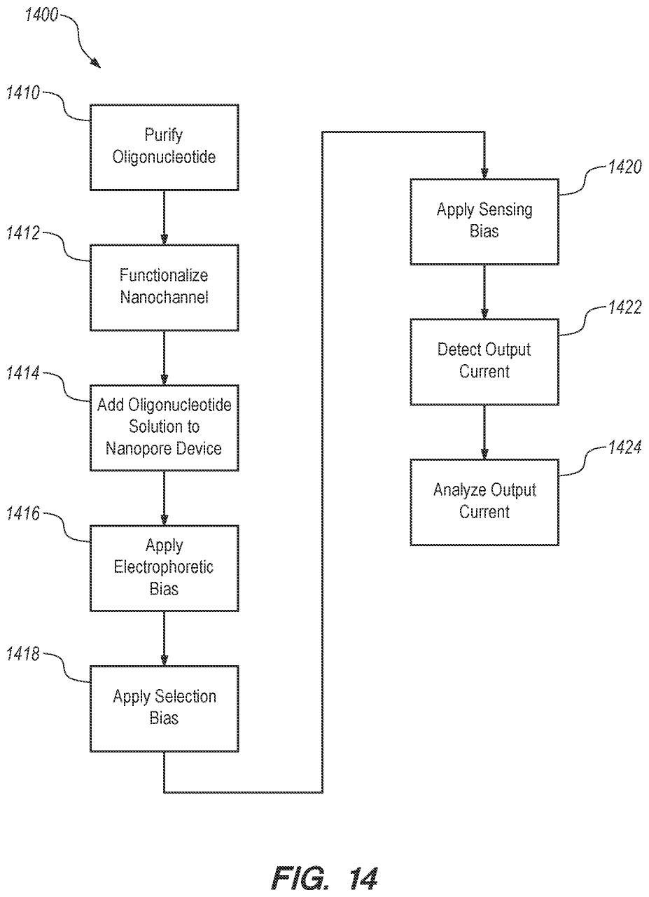

With reference data such as that depicted in FIG. 13, the nanopore detection systems described herein can be used in a method of detecting methylation of oligonucleotides. For instance, FIG. 14 depicts a method 1400 of detecting methylation of oligonucleotides using a nanopore detection system according to some embodiments. At step 1410, a target oligonucleotide is purified. The target oligonucleotide may be a CpG island in a promoter of a gene (e.g., a cancer suppressing gene).

At step 1412, a nanochannel is functionalized. The nanochannel is in a 3D nanopore device having top and bottom chambers, with the a 3D nanochannel array disposed in the top and bottom chambers such that the top and bottom chambers are fluidly coupled by a plurality of nanochannels in the 3D nanochannel array. The nanochannel may be functionalized by coupling an oligonucleotide probe to an inner surface of the 3D nanopore device defining the nanochannel, wherein the oligonucleotide probe is complementary to the oligonucleotide.

At step 1414, a DI water solution with the oligonucleotide is added to the 3D nanopore device.

At step 1416, an electrophoretic bias is applied to top and bottom electrodes in the top and bottom chambers of the 3D nanopore device to drive charged particles through the nanochannels.

At step 1418, a selection bias is applied to first and second gating nanoelectrodes in the 3D nanopore device to direct flow of the oligonucleotide through a nanochannel of a plurality of nanochannels in the 3D nanopore device.

At step 1420, a sensing bias is applied to a sensing electrode in the 3D nanopore device to elicit an output current.

At step 1422, an output current is detected from the sensing electrode.

At step 1424, the output current from the sensing nanoelectrode to determine a methylation percentage of the oligonucleotide. For instance, the output current can be compared to reference data such as that depicted in FIG. 13. Taking multiple output current measurements while changing/sweeping the sensing bias applied to the 3D nanopore device can improve the accuracy of methylation percentage determination.

The nanopore detection systems described herein are 3D sensors that work with DI water as a buffer. The function and exact mechanism of action for water molecules within nanoscale small spaces have not been previous investigated and understood, but highly sensitive and clear resolution of the 3D arrays described herein may prove the benefit of using DI water instead of electrolytes or other buffer solutions, which increases the noise level within such sensitive sensors.

The mechanism of reaction and signal generation in the nanopore detection systems described herein is based on changing the charge distribution in the surface because of hydration of methylated DNA molecules that attach to the probes described above. This hydration causes changes in the electrode with the redistribution of charge density at the gate nanoelectrodes. Nanoelectrodes inside of the nanopores have an all-around or belt-like morphology surrounding the nanopore, which increases the sensitivity of the nanopore sensor.

By using different potential gradients at each nanopore, a user can control the speed of charged biomolecules traveling inside and through each nanopore. Using a low concentration buffer/electrolyte or DI water to increase the Debye length of the sensing area in the nanopore is one of the unique properties of the 3D nanopore detection systems described herein. A user has broad control over the nanopore detection system by changing the amount and duration of electrical potential for each nanoelectrode to electrophoretically control movement of the charged target biopolymers and the Ping-Ponging motion of same between the nanoelectrodes as described above. As described above, when charged target biopolymers moves back and forth between nanoelectrodes with changing/alternating nanoelectrode potential, time required for the charged target biopolymers to attach to the probes will be reduces to less than 10 minutes. This reduction in attachment time is due to increased interaction between the targets and the probes, allowing them to bond with each other in less time.

In some embodiments of nanopore detection systems, such as those described herein, the size, shape, and depth of the nanopore structure can be modified based on the size of the probe. For instance, a pore size with a diameter of 50 nm (500 Å) may be used for sensing target biopolymers with a 40 bp probe. In other embodiments, a pore size with a diameter of 100 nm may use for sensing target biopolymers with more than 100 bp probes. In still other embodiments, a pore size with a diameter of 200 nm may be used for sensing target biopolymers with still longer probes.

The 3D nanopore array sensors described herein are more sensitive and compact compared to 2D or planar structure sensors because the 3D array of nanopores increases the surface to volume ratio, allowing for miniaturization of the smart surfaces inside the nanochannels of the nanopore arrays. The high surface to volume ratio allows sensing of very low concentrations (e.g., 10 femtomolar) of DNA methylation.

The 3D nanopore array sensors described herein provide better control compared to charge perturbation or electrochemical based sensor systems because the dielectric layer insolates the inner surfaces of each nanochannel, thereby enhancing the capacitance effect and control of the electrical field effect for each nanochannel.

The 3D nanopore array sensors described herein can use capacitance variation for sensing DNA methylation with an immobilized probe. When a target DNA molecule passes within a nanopore of the array structure (electrophoretically driven by the external voltage), the top and bottom electrodes record a change in the potential resulting from the passing DNA molecule within the nanopore structure, polarizing the nanopore like a capacitor. The resulting capacitance variation can be measured electronically to detect passage of the target DNA molecule. The speed of the DNA molecule can be controlled by controlling the applied positive gate biases, allowed the 3D nanopore array sensor to be used in methylation detection. The 3D nanopore array sensors described herein can detect passage of DNA methylation by detecting both tunneling current and capacitance change. Previously existing biological nanopores cannot detect tunneling current and capacitance change because they do not have embedded nanoelectrodes in their structure.

The probes used in the 3D nanopore array sensors described herein may be modified to alter their surface chemistry, allowing more system control and design options. For instance, thiol modification may be used for thiol gold binding. Avidin/biotin and EDC crosslinker/N-hydroxysuccinimide (NHS) are other probe modification and target pairs that may be used with the 3D nanopore array sensors described herein with modification of structure and chemistry of immobilizing techniques.

The corresponding structures, materials, acts and equivalents of all means or step plus function elements in the claims below are intended to include any structures, materials, acts and equivalents for performing the function in combination with other claimed elements as specifically claimed. It is to be understood that while the invention has been described in conjunction with the above embodiments, the foregoing description and claims are not to limit the scope of the invention. Other aspects, advantages and modifications within the scope to the invention will be apparent to those skilled in the art to which the invention pertains.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. Other details of the present invention, these may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A method of determining an oligonucleotide methylation percentage, comprising:
providing a 3D nanopore device having top and bottom chambers, and a 3D nanochannel array disposed in the top and bottom chambers such that the top and bottom chambers are fluidly coupled by a plurality of nanochannels in the 3D nanochannel array;
purifying an oligonucleotide;
functionalizing the 3D nanochannel array by coupling an oligonucleotide probe to an inner surface of the 3D nanopore device defining the nanochannel, wherein the oligonucleotide probe is complementary to the oligonucleotide;
adding the purified oligonucleotide to DI water to form an oligonucleotide solution having a known concentration;
adding the oligonucleotide solution including the oligonucleotide to the top and bottom chambers;
placing top and bottom electrodes in the top and bottom chambers respectively;
applying an electrophoretic bias between the top and bottom electrodes;
applying a selection bias across first and second gating nanoelectrodes in the 3D nanopore device to direct flow of the oligonucleotide through a nanochannel of the plurality of nanochannels;
applying a sensing bias through a sensing nanoelectrode in the 3D nanopore device;
detecting an output current from the sensing nanoelectrode; and
analyzing the output current from the sensing nanoelectrode to determine a methylation percentage of the oligonucleotide.

2. The method of claim 1, further comprising functionalizing the 3D nanochannel array by coupling a second oligonucleotide probe to an inner surface of the 3D nanopore device defining a second nanochannel,
wherein the second oligonucleotide probe is different from the oligonucleotide probe.

3. The method of claim 1, wherein analyzing the output current from the sensing electrode to determine a methylation percentage of the oligonucleotide comprises comparing the output current and the sensing bias to corresponding values in a reference table for the known concentration.

4. The method of claim 1, wherein analyzing the output current from the sensing electrode to determine a methylation percentage of the oligonucleotide comprises using an effect of methylation on a charge of a phosphate backbone of the oligonucleotide.

5. The method of claim 1, further comprising:
applying a second sensing bias through the sensing nanoelectrode in the 3D nanopore device;
detecting a second output current from the sensing nanoelectrode;
analyzing the second output current from the sensing nanoelectrode to determine a second methylation percentage of the oligonucleotide; and
comparing the second methylation percentage of the oligonucleotide to the methylation percentage of the oligonucleotide to confirm the methylation percentage of the oligonucleotide.

6. The method of claim 1, wherein the oligonucleotide is an RNA molecule fragment or a DNA molecule fragment.

7. (canceled)

8. The method of claim 1, wherein the oligonucleotide is extracted from cell free DNA, tissue or cell culture medium, serum, urine, plasma, or saliva.

9.-10. (canceled)

11. The method of claim 1, wherein charge carriers in the 3D nanopore device comprise the DI water, H+ ions, and OH− ions.

12. The method of claim 1, further comprising:
removing the oligonucleotide solution including the oligonucleotide from the top and bottom chambers;
purifying a second oligonucleotide;
functionalizing the 3D nanochannel array by coupling a second oligonucleotide probe to an inner surface of the 3D nanopore device defining the nanochannel, wherein the second oligonucleotide probe is complementary to the second oligonucleotide;
adding the purified second oligonucleotide to DI water to form a second oligonucleotide solution having a known concentration;
adding the second oligonucleotide solution including the second oligonucleotide to the top and bottom chambers;
applying the electrophoretic bias between the top and bottom electrodes;
applying the selection bias across the first and second gating nanoelectrodes in the 3D nanopore device to direct flow of the second oligonucleotide through the nanochannel;
applying the sensing bias through the sensing nanoelectrode in the 3D nanopore device;
detecting a second output current from the sensing nanoelectrode; and
analyzing the second output current from the sensing nanoelectrode to determine a methylation percentage of the second oligonucleotide.

13. The method of claim 1, further comprising:
applying a second selection bias across third and fourth gating nanoelectrodes in the 3D nanopore device to direct flow of a second oligonucleotide through a second nanochannel of the plurality of nanochannels;
applying a second sensing bias through a second sensing nanoelectrode in the 3D nanopore device;
detecting a second output current from the second sensing nanoelectrode; and
analyzing the second output current from the second sensing nanoelectrode to determine a methylation percentage of the second oligonucleotide.

14. The method of claim 1, wherein analyzing the output current from the sensing electrode to determine a methylation percentage of the oligonucleotide comprises differentiating between methyl cytosine methylation and hydroxy methyl cytosine methylation.

15. The method of claim 1, further comprising comparing the methylation percentage of the oligonucleotide to a library of methylation patterns corresponding to known mutations to diagnose a disease,
wherein the disease is cancer, atherosclerosis, or aging.

16. (canceled)

17. The method of claim 1, wherein the oligonucleotide probe is a DNA probe, an RNA probe, or a protein probe.

18. The method of claim 1, further comprising analyzing the output current from the sensing nanoelectrode to quantify a number of methylation sites in the oligonucleotide.

19. The method of claim 1, further comprising applying a rate control bias to a rate control nanoelectrode in the 3D nanopore device to modulate a translocation rate of the oligonucleotide through the nanochannel.

20. The method of claim 1, wherein the current is an electrode current.

21. The method of claim 1, wherein the current is a tunneling current.

22. The method of claim 1,
wherein the first gating nanoelectrode addresses a first end of the nanochannel,
wherein the second gating nanoelectrode addresses a second end of the nanochannel opposite the first end, and
wherein a sensing nanoelectrode addresses a first location in the nanochannel between the first and second ends.

23. The method of claim 1, further comprising alternatively reversing the electrophoretic bias and the selection bias to direct alternating flow of the oligonucleotides through the nanochannel between the first and second gating nanoelectrodes.

24. The method of claim 1, wherein the 3D nanopore device is integrated into a mobile application, a laptop computer, or a desktop computer.

25. The method of claim 1, wherein the 3D nanopore device is integrated into microfluidic device, a nanofluidic device, a nanodevice, or a lab-on-chip system.

26. The method of claim 1, wherein the 3D nanopore device is integrated into an all-in-one ASIC platform system for extraction and sensing of the oligonucleotide.

27. The method of claim 1, further comprising:
the 3D nanopore device detecting hybridization of the oligonucleotide to the oligonucleotide probe at a minimum concentration of the oligonucleotide of about 10 femtomolar (limit of detection); and
the 3D nanopore device detecting hybridization of the oligonucleotide to the oligonucleotide probe without amplification of the oligonucleotide or use of PCR,
wherein the 3D nanopore device is integrated into a liquid biopsy panel platform to perform detection without amplification of the oligonucleotide or use of PCR.

28.-29. (canceled)

30. The method of claim 1, further comprising analyzing the output current from the sensing nanoelectrode to determine a conformation change of the oligonucleotide.

31. The method of claim 1, further comprising analyzing the output current from the sensing nanoelectrode to determine a hydration change of the oligonucleotide.

32.-33. (canceled)
